(19) United States
(12) Patent Application Publication
(10) Pub. No.: US 2026/0232377 A1
(43) Pub. Date: Aug. 13, 2026

(54) LASER EMISSION MODULATION FOR TREATMENT OF CALCULI

(71) Applicant: IPG PHOTONICS CORPORATION, Marlborough, MA (US)

(72) Inventors: Gregory ALTSHULER, Lincoln, MA (US); ILYA YAROSLAVSKY, North Andover, MA (US); James CHILDS, Bolton, MA (US); Anastasia KOVALENKO, Wellesley, MA (US); Dilip PAITHANKAR, Waltham, MA (US)

(73) Assignee: IPG PHOTONICS CORPORATION, Marlborough, MA (US)

(21) Appl. No.: 19/149,177

(22) PCT Filed: Jan. 19, 2024
(86) PCT No.: PCT/US2024/012187

§ 371 Date: Jul. 18, 2025

Related U.S. Application Data
(60) Provisional application No. 63/440,191, filed on Jan. 20, 2023.

(51) Int. Cl. A61B 18/26; A61B 18/00

(52) U.S. Cl. CPC A61B 18/26; A61B 2018/00702; A61B 2018/00726; A61B 2018/00761

(57) ABSTRACT

Laser-based systems and method for treatment of calculi. The system can include a laser configured to emit pulsed laser energy and a controller configured to control the laser. The laser can be modulated to operate in one of a dusting, fragmentation, or reduced retropulsion operational mode. One or more of these operational modes include pulses comprised of sub-pulses.

### RELATED APPLICATIONS

The present application claims priority to U.S. Provisional Application Ser. No. 63/440,191, titled “LASER EMISSION MODULATION FOR TREATMENT OF HARD TISSUE,” filed on Jan. 20, 2023, the content of which is hereby incorporated by reference in its entirety.

### BACKGROUND

### Technical Field

The technical field relates generally to laser treatment of calculi, and more specifically to modulated pulsed laser energy in the treatment of calculi.

### Background Discussion

Kidney stone disease is a prevalent condition estimated to be affecting 12% of the world population. Although most of the patients can pass the stones naturally, the condition can be severe enough that would require medical intervention. Extreme pain, nausea, vomiting, infection, blockage of urine flow and loss of kidney function can follow. Laser lithotripsy is a method for treatment of kidney stones where light energy directed by an optical fiber is used to break the stone into finer parts that can be passed naturally.

Optimal targeting of calculi such as stones and minimizing the risk of accidental damage to surrounding intact soft tissues are two major problems of laser lithotripsy. There is therefore a need for a technical solution that addresses both these problems through optimizing the temporal structure of the laser output.

Until recently, relevant laser sources (e.g., Ho:YAG laser) had been extremely limited in their ability to be modulated. However, the advent of newer laser sources (e.g., diode laser pumped Tm fiber laser and Tm: YAG laser) offer the possibility to modulate the laser across a wider range of outputs.

### SUMMARY

Aspects and embodiments are directed to methods and systems of modulating laser emission for treatment of calculi using pulsed laser energy.

In accordance with an exemplary embodiment, a laser system for treating calculi is provided that includes a laser configured to emit pulsed laser energy having a wavelength in a range of 1.85 to 2.2 microns (μm) inclusive, and a controller configured to control the laser such that the pulsed laser energy is emitted as a sequence of sub-pulse groups separated in time by a pulse repetition interval, and each sub-pulse group includes at least two sub-pulses separated in time by a sub-pulse interval that is in a range of 0-10 milliseconds (ms), wherein each sub-pulse of the sub-pulse group has a pulse duration in a range of 0.001-5 ms inclusive, each sub-pulse of the sub-pulse group has an energy in a range of 0.001-1 Joules (J) inclusive, and the pulse repetition interval is in a range of 1-1000 ms inclusive.

In accordance with another exemplary embodiment, a method for treating calculi is provided that includes generating pulsed laser energy having a wavelength in a range of 1.85 to 2.2 microns (μm) inclusive, emitting the pulsed laser energy as a sequence of sub-pulse groups separated in time by a pulse repetition interval, where each sub-pulse group includes at least two sub-pulses separated in time by a sub-pulse interval that is in a range of 0-10 milliseconds (ms)inclusive, each sub-pulse of the sub-pulse group has a pulse duration in a range of 0.001-5 milliseconds (ms) inclusive, each sub-pulse of the sub-pulse group has an energy in a range of 0.001-1 Joules (J) inclusive, and the pulse repetition interval is in a range of 1-1000 ms inclusive, and directing the pulsed laser beam at a treatment area of calculi.

In one example, the sub-pulse group includes 2-1000 sub-pulses. In another example, the sub-pulse group includes 2-100 sub-pulses. In another example, the sub-pulse group includes 2-10 sub-pulses.

In one example, a total energy of the sub-pulse group is in a range of 0.2-5 J inclusive. In a further example, the total energy of the sub-pulse group is in a range of 0.5-2 J inclusive.

In one example, the energy of each sub-pulse is in a range of 0.01-1 J inclusive. In a further example, the energy of each sub-pulse is in a range of 0.2-1 J inclusive.

In one example, the sub-pulse interval is in a range of 0.01-5 ms inclusive. In a further example, the sub-pulse interval is in a range of 0.01-1 ms inclusive.

In one example, a duration of each sub-pulse is in a range of 0.1-1 ms inclusive.

In one example, the pulsed laser energy has an average power in a range of 2-120 Watts (W) inclusive. In another example, the average power is in a range of 2-40 W inclusive. In another example, the average power is in a range of 5-40 W inclusive.

In one example, the pulse repetition rate within the sub-pulse group is in a range of 0.5-500 Hz inclusive.

In one example, a peak power of a sub-pulse of the sub-pulse group is in a range of 250-20000 W inclusive. In another example, the peak power of the sub-pulse of the sub-pulse group is in a range of 500-5000 W inclusive.

In one example, at least one sub-pulse of the sub-pulse group has a pulse shape such that a power of the at least one sub-pulse monotonically increases from a beginning of the sub-pulse to an end of the sub-pulse.

In one example, the laser is a thulium fiber laser, a thulium solid state laser, or a holmium solid state laser.

In one example, a size of a fragment of the calculi after being exposed to the pulsed laser beam is less than 1 mm in a maximum cross-sectional dimension.

In accordance with another exemplary embodiment, a laser system for treating calculi is provided that includes a laser configured to emit pulsed laser energy having a wavelength in a range of 1.85 to 2.2 microns (μm) inclusive, and a controller configured to control the laser such that each pulse of the emitted pulsed laser energy comprises two groups of sub-pulses separated in time by a sub-pulse group interval, wherein each group of sub-pulses includes at least two sub-pulses separated in time by a sub-pulse interval that is in a range of 0-1 millisecond (ms) inclusive, the first group of sub-pulses has sufficient total energy, total pulse length, and an average power to generate mechanical stress in a target calculi, the second group of sub-pulses has sufficient total energy, total pulse length, and an average power to generate a thermo-mechanical impact in the target calculi, wherein a total energy of the two groups of sub-pulses is in a range of 2-70 Joules (J) inclusive, and a repetition rate of the two groups of sub-pulses is in a range of 0.5-10 Hertz (Hz) inclusive.

In accordance with another exemplary embodiment, a method for treating calculi is provided that includes generating pulsed laser energy having a wavelength in a range of 1.85 to 2.2 microns (μm) inclusive, emitting the pulsed laser energy as two groups of sub-pulses separated in time by a sub-pulse interval in a range of 0-1 milliseconds (ms) inclusive and configured such that the first group of sub-pulses has sufficient total energy, total pulse length, and an average power to generate mechanical stress in a target calculi, the second group of sub-pulses has sufficient total energy, total pulse length, and an average power to generate a thermo-mechanical impact in the target calculi, a total energy of the two groups of sub-pulses is in a range of 2-70 Joules (J) inclusive, and a repetition rate of the two groups of sub-pulses is in a range of 0.5-10 Hertz (Hz) inclusive, and directing the pulsed laser beam at the target calculi.

In one example, a size of a fragment of the target calculi after being exposed to the pulsed laser energy is at least 1 mm in a maximum cross-sectional dimension. In another example, a size of a fragment of the target calculi after being exposed to the pulsed laser energy is in a range of 1-5 mm in a maximum cross-sectional dimension. In another example, a size of a fragment of the target calculi after being exposed to the pulsed laser energy is in a range of 1-3 mm in a maximum cross-sectional dimension.

In one example, an energy of the first group of sub-pulses is in a range of 1-65 J inclusive. In another example, the energy of the first group of sub-pulses is in a range of 6.5-65 J inclusive.

In one example, an energy of the second group of sub-pulses is in a range of 1-6.5 J inclusive.

In one example, the target calculi has an ablation threshold and the energy of the first group of sub-pulses does not exceed the ablation threshold. In another example, the target calculi has an ablation threshold and the energy of the first group of sub-pulses is less than the ablation threshold multiplied by a factor of 1.5.

In one example, an average power of the first group of sub-pulses is in a range of 50-200 Watts (W) inclusive.

In one example, the first group of sub-pulses comprises at least two sub-pulses separated in time by a sub-pulse interval. In another example, the sub-pulse interval is of a duration to prevent fluid penetration of an area of the target calculi that has been exposed to the pulsed laser energy.

In one example, the first group of sub-pulses includes 1-100 sub-pulses.

In one example, a peak power of the second group of sub-pulses is in a range of 500-20,000 W inclusive. In another example, the peak power of the second group of sub-pulses is in a range of 500-1500 W inclusive.

In one example, the laser is a thulium fiber laser or a solid state laser.

Still other aspects, embodiments, and advantages of these example aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Embodiments disclosed herein may be combined with other embodiments, and references to “an embodiment,” “an example,” “some embodiments,” “some examples,” “an alternate embodiment,” “various embodiments,” “one embodiment,” “at least one embodiment,” “this and other embodiments,” “certain embodiments,” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1A is a chart showing one example of a pulse sequence optimized for a stone dusting procedure in accordance with one or more aspects of the invention;

FIG. 1B is a chart showing another example of a pulse sequence optimized for a stone dusting procedure in accordance with one or more aspects of the invention;

FIG. 1C is a chart showing another example of a pulse sequence optimized for a stone dusting procedure in accordance with one or more aspects of the invention;

FIG. 2A is a chart showing one example of a pulse sequence optimized for a stone fragmentation procedure in accordance with one or more aspects of the invention;

FIG. 2B is a chart showing another example of a pulse sequence optimized for a stone fragmentation procedure in accordance with one or more aspects of the invention;

FIGS. 3A and 3B are images taken from a high speed video of a stone ablation procedure and show a distribution and quantity of a product of ablation between the stone and the distal end of the fiber in accordance with aspects of the invention;

FIG. 4 is three images taken from a high speed video of a stone ablation procedure and show heat radiation at the distal end of the fiber in accordance with aspects of the invention;

FIG. 5 is a chart showing an example of transmission oscillations over time in accordance with aspects of the invention;

FIG. 6 is a chart showing an example of the number of transmission oscillations over time in accordance with aspects of the invention;

FIG. 7A is chart showing one example of a pulse optimized for reducing retropulsion in accordance with prior art;

FIG. 7B is a chart showing one example of a pulse sequence optimized for reducing retropulsion in accordance with one or more aspects of the invention;

FIG. 7C is a chart showing another example of a pulse sequence optimized for reducing retropulsion in accordance with one or more aspects of the invention;

FIG. 8 is a chart showing an example of a pulse profile in accordance with aspects of the invention;

FIG. 9 is a chart showing one example of a pulse configuration in accordance with aspects of the invention; and

FIG. 10 is a block diagram of a laser system for treating calculi in accordance with one or more aspects of the invention.

### DETAILED DESCRIPTION

In accordance with one or more embodiments, particular systems and methods of modulating laser emission are considered below for specific purposes of treating calculi. As used herein, the term “calculi” refers to calculi (stones) present in anatomical locations, such as the ureter, kidney, or bladder. Calculi includes all types of stones in a human or animal body. One or more aspects of this disclosure can be used for diode pumped Thulium (Tm) and Holmium (Ho) doped crystal or fiber lasers with output wavelengths in the range between 1.85 and 2.2 μm inclusive. In some embodiments, a Tm fiber laser or Tm:YAG laser may be employed. In some embodiments, a solid state laser may be used, such as Tm:YAG or Ho:YAG. In accordance with various embodiments, a thulium fiber laser, a thulium solid state laser, or a holmium solid state laser may be used in the system and methods described herein.

### Maximizing Efficiency of Stone Dusting Procedures (Dusting Operational Mode)

The primary goal of stone dusting procedures is to produce the smallest stone fragments possible within the shortest period of time. The clinical goal of dusting is to break stones into particles with sizes smaller than 1 mm (preferably smaller than 0.25 mm, which is considered “fine dusting”) to guarantee natural evacuation of all fragments within several weeks after surgery to prevent new stone formation. In accordance with the dusting modes described herein, according to at least one embodiment a size of a fragment of the calculi after being exposed to the pulsed laser beam is less than 1 mm in a maximum cross-sectional dimension (if the fragment is circular, then this would be a diameter), and in some embodiments is less than 0.25 mm in the maximum cross-sectional dimension. Conventional laser dusting procedures utilize regular pulses which are generated by a laser that is not configured with any kind of special modulation. For example, a regular pulse generated by a thulium fiber laser, which is widely used in clinical practice, has a rectangular or flat-top pulse shape. Typically, these pulses have spurious modulation associated with relaxation oscillation manifesting itself as spikes with pulse lengths 0.01-5 microseconds (μs). However, a typical energy of these spikes is below the threshold of calculi ablation and the effects of these spikes are negligible. The pulse structures and pulse shapes as disclosed and described herein will be defined without regard to the presence of such spikes.

At a fixed average power, the use of conventional high pulse energy/low repetition rate operational modes results in relatively large fragments that have to be broken down even further. In the alternative, the use of conventional low pulse energy/high repetition rate operational modes creates suitably small fragments, but lacks in stone ablation efficiency and therefore leads to increased procedure times. Both of these outcomes are sub-optimal.

In accordance with certain embodiments, two mechanisms of dusting are proposed.

(1) Adiabatic vaporization of water in the interspace that exists between microcrystals forming the stone as well as in clusters of the microcrystals and in stone pores and other stone defects that are filled by water. Vapor pressure due to water vaporization initiates stone fragmentation into microcrystals having a size in a range of 0.01-50 μm inclusive, or into clusters having a size within a range of 10-1000 μm inclusive. To maximize efficiency of this mechanism, the pulse width in some embodiments is shorter than the thermal relaxation time of inter micro crystal or inter cluster space filled by water (thermally confined) and preferably shorter than the relaxation time of vapor pressure (acoustically confined). In accordance with at least one embodiment, the laser pulse energy and fluence are higher than the water vaporization energy threshold.
(2) Microchipping (delamination) of small fragments from the bottom of the laser crater caused by a thermomechanical gradient of pressure perpendicular to the surface of the laser crater bottom. The thickness of the microchips is approximately equal to the penetration depth of laser light in stone that is defined by the absorption coefficient of water. The pulse width (pulse duration) should be shorter than the thermal relaxation time of a layer of stone having a thickness corresponding to the penetration depth. Another mechanism of laser-induced delamination of small fragments from the bottom of the laser crater can be due to formation of a dry layer of the stone after a laser pulse, penetration of the energy of the subsequent pulse through this layer, and ablation of the wet stone layer under the dry layer.

The laser systems and methods as disclosed herein are based on a discovery by Applicant that the laser pulse structure modulation provides substantial improvement in the speed of dusting and reduction of the mean fragment size. The discovery was done as a result of comparing various pulse structures and shapes generated by the laser with modulated pulse structures using preclinical and clinical settings and standard experimental methodology. Pulse structures and shapes, the ranges of energy, power and pulse intervals, pulse length and other characteristics were defined based on a substantial (more than 25%) improvement in comparison with the existing (conventional) regular pulse treatments. In accordance with at least one embodiment, pulses structured as sequences of relatively low-energy or optimal sub-pulses (micropulses) are used to perform dusting procedures. In some embodiments, the sub-pulses or micropulses are implemented with short intervals in between the sub-pulses to prevent cooling of the stone via water penetration in between the sub-pulses. As used herein, the term “sub-pulse” or “micropulse” refers to short pulses, a multitude of which together form a resulting pulse. In one embodiment, the sequences of low-energy sub-pulses are combined into groups of 2-1000, in another embodiment into groups of 2-100, and in yet another embodiment into group of 2-10 pulses. According to at least one embodiment, a size of the calculi is less than 0.25 mm in diameter.

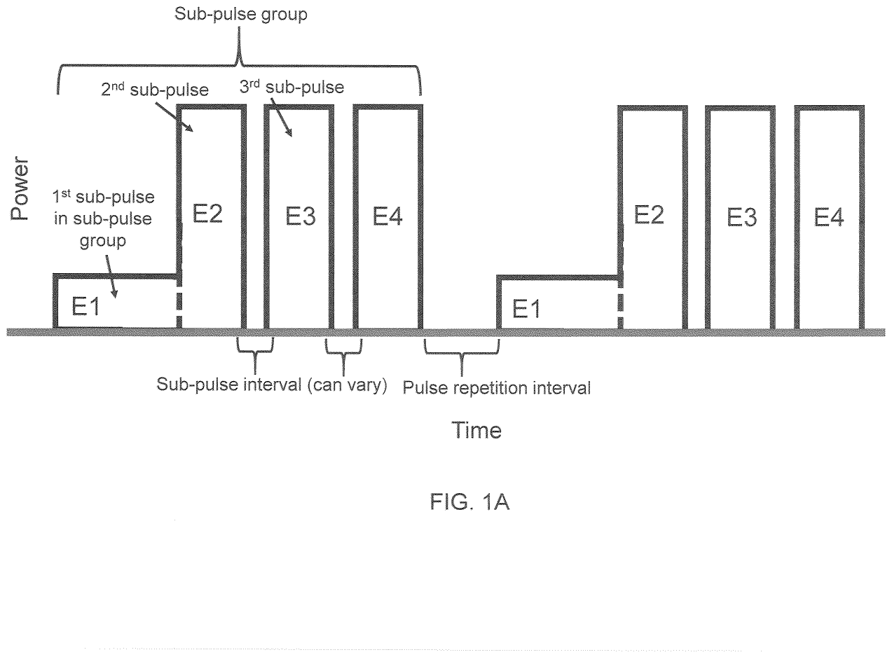

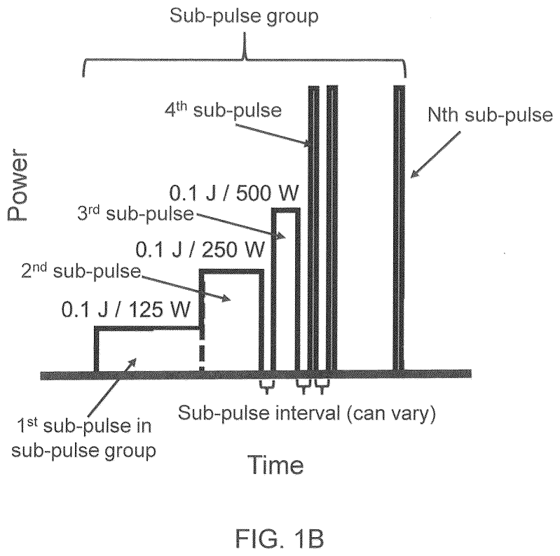

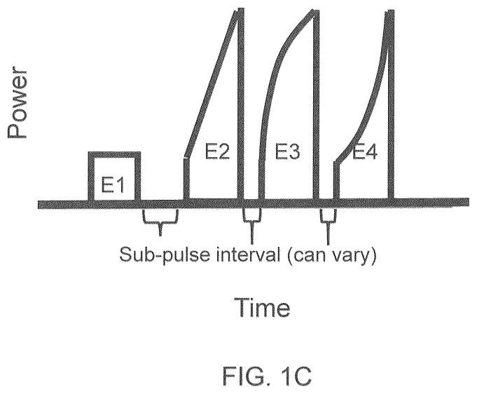

One non-limiting example of a two sub-pulse groups used in a pulse sequence for stone dusting is shown in FIG. 1A. The term “sub-pulse group” refers to several sub-pulses which, when transmitted, are separated in time (from one another) by a sub-pulse interval. Sub-pulse groups are separated in time by a pulse repetition interval. In accordance with at least one embodiment, the sub-pulse group is initiated by one or more lower-peak power sub-pulses for purposes of minimizing the retropulsion effect, i.e., when stone fragments migrate away from the energy source. In some embodiments, the peak power may be increased after the initial 1-3 pulses. The presence of multiple low-energy short micropulses creates sufficiently small fragments, while the high cumulative energy created by the pulse sequence delivered in a relatively short time interval ensures high ablation efficiency. After ablation by each micropulse, the bottom of the laser crater is covered by a thin layer of dry stone with vaporized water. This layer can then be removed as microchipping or delamination of the stone material on the bottom of the laser crater by the next pulse due to ablation of the wet stone material under the dry layer. For high ablation efficiency it is critical to keep the interval between pulses below the penetration time of water in the dry layer that occurs between micropulses and cools the stone material. According to some embodiments, the sub-pulse interval is in a range of 0-10 ms inclusive, in some embodiments is in a range from 0.01-5 ms inclusive, in some embodiments is in a range from 0.01-1 ms inclusive, in some embodiments is in a range from 0.01-0.2 ms inclusive, and in some embodiment is in a range of 0.01-1 ms inclusive. In accordance with certain embodiments, one or more of the sub-pulse intervals in a sub-pulse group may vary. For example, in FIG. 1A the first sub-pulse group includes four sub-pulses, where the sub-pulse interval is 0 between the first sub-pulse and the second sub-pulse (meaning the first and second sub-pulses are joined, as shown in FIG. 1A), and this first sub-pulse interval is a different value than the duration of the other sub-pulse intervals in the sub-pulse group.

Some embodiments may include variations of the base sequence of FIG. 1A. For example, the first few pulses in the sequence may have gradually increasing amplitude (peak power) in order to further minimize retropulsion and shift the residual fragments size distribution toward smaller particles. One non-limiting example of such a pulse group configured in such a sequence is shown in FIG. 1B, which is another example of a pulse sequence that may be used for a stone dusting procedure.

An additional example of a pulse sequence that may be used for a dusting procedure is shown in FIG. 1C. According to at least one embodiment, at least one sub-pulse of the sub-pulse group has a pulse shape such that a power of the at least one sub-pulse monotonically increases from a beginning of the sub-pulse to an end of the sub-pulse. The second, third, and fourth pulses of the sub-pulse group in FIG. 1C are non-limiting examples of such a power profile with a monotonically increasing function. This configuration is and other examples are discussed in further detail below.

Returning to FIG. 1C as an example, each sub-pulse can be optimized to be configured with a pulse shape to maximize an effect of stone ablation with dust formation. Applicant discovered in experimental work using high speed photography that attenuation of the laser beam was increasing during the laser pulse and this effect was increasing with the peak power of a rectangular pulse. There are two mechanisms that can explain this phenomenon.

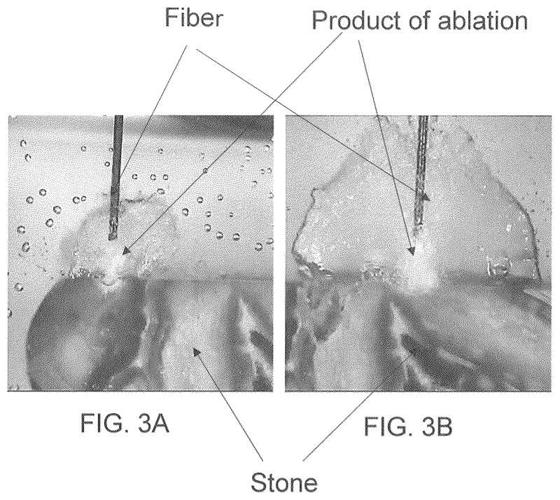

The first mechanism is due to increase in the scattering and absorption of the laser beam on the flow of the products of ablation (effect of screening the laser crater) with the increase of the laser power. In FIGS. 3A and 3B two video frames are shown of a crater of ablation and the flow of the products of ablation (i.e., a flow of particles-dust) of COM stone exposed with a rectangular laser pulse generated by a Thulium Fiber Laser with a wavelength 1.94 μm, an energy 0.5 J, and a peak power of 250 W (FIG. 3A) and a peak power of 2000 W (FIG. 3B) with particular interest shown at the end of laser pulse. One can see from the picture that angle of the flow of ablation particles for low peak power (FIG. 3A) is larger than for higher peak power (FIG. 3B) but the amount and density of particles and beam scattering and absorption is lower for 250 W (FIG. 3A) in comparison with 2000 W (FIG. 3B).

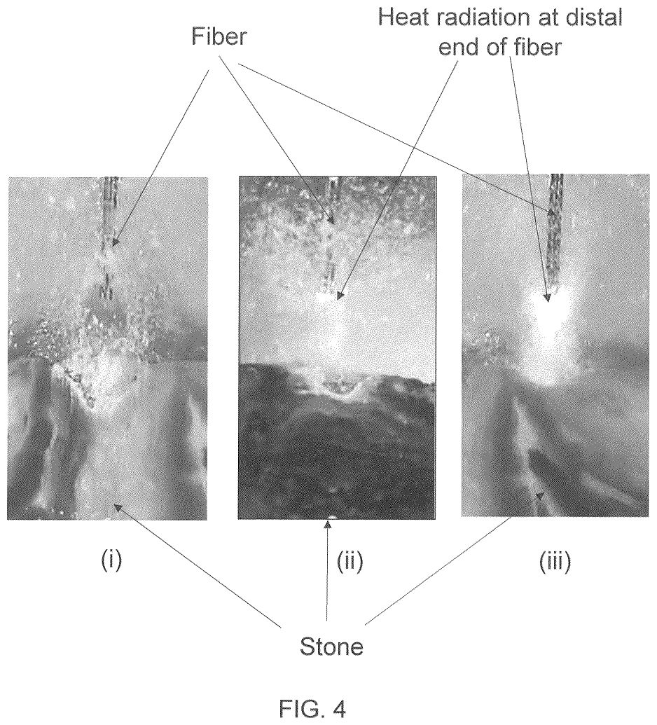

The second mechanism discovered is illustrated by FIG. 4, which shows three frames ((i), (ii), and (iii)) of a video captured with a high speed camera and includes a video frame of the crater of ablation and condition of the fiber tip that was used to deliver laser energy of the same fiber as FIGS. 3A and 3B (i.e., a Thulium Fiber Laser directed at COM stone) but with a peak power of 500 W (i), 1000 W (ii), and 2000 W (iii) at the end of a rectangular pulse and with an energy of 2 J. The figures indicate that the fiber tip is emitting visible light, which is heat radiation of the fiber tip heated to a high temperature due to the initial absorption of the laser energy by products of ablation that have contaminated the distal end of the fiber tip. The intensity of such heat radiation in the visible spectrum increases with the temperature of the tip. The images shown in FIG. 4 indicate that there is no heat radiation from the fiber tip for the 500 W peak power (i), there is low heat radiation for 1000 W (ii), and there is very intense heat radiation for 2000 W (iii). The temperature of the tip at the end of the pulse for 500 W is below 400° C., for 1000 W is 600-900° C., and for 2000 W it is above 1300° C. It is known that heating of the quartz fiber to temperatures greater than 800° C. initiates an effect in the quartz material that sharply increases its absorption. That, in turn, increases the attenuation of laser energy being delivered to the stone for ablation and decreases the ablation efficiency. Normally, increasing the peak power or rectangular pulse with a given energy results in an increase in ablation efficiency, but the additive impact of the two discovered mechanisms can nullify or even reverse this effect. When comparing (i), (ii), and (iii) of FIG. 4, it is apparent that the size of the crater for the higher peak power (iii) is smaller than for the lower peak power (i) for the equal laser energy of 2 J. Due to the combination of such mechanisms for every given condition (such as stone local composition and structure, laser pulse energy, fiber diameter, distance between fiber tip and stone) there should be an optimum peak power, which provides the maximum ablation efficiency. Since this optimum peak power depends on different factors that can vary during stone treatment, according to at least one embodiment, the pulse shapes with varying power during the pulse within a certain range to guarantee that this range contains the optimal peak powers for various locations of stone surface, varying gaps, and other variables during treatment.

According to at least one embodiment, at least one sub-pulse of the sub-pulse group has a pulse shape such that a power of the at least one sub-pulse increases from a beginning of the sub-pulse to an end of the sub-pulse. In accordance with various embodiments, this increase can be either monotonic or non-monotonic. The increase can follow one or more functions, for example, a step function, linear, polynomic or exponential function. The second, third, and fourth pulses of the sub-pulse group in FIG. 1C are non-limiting examples of such a power profile with a monotonically increasing function. In some embodiment, the energy of such sub-pulse can be in the range of 0.2-1 J inclusive. In some embodiments the interval between such sub-pulses is in the range 0.01-5 ms inclusive for purposes of preventing water penetration into the laser crater between sub-pulses.

In accordance with at least one embodiment, optimal laser parameters for a dusting operational mode using pulse modulation as described above are as follows:
Energy of each sub-pulse: 0.001-1 J inclusive, in some embodiments is 0.01-1 J inclusive, in further embodiments is 0.2-1 J inclusive, and in some embodiments the sub-pulse energy may vary within a sub-pulse group, e.g., a first sub-pulse may have lower energy than a second sub-pulse in the sub-pulse group;
Total energy of pulse (energy of the group of sub-pulses): 0.2-5 J inclusive, preferably 0.5-2 J inclusive;
Pulsewidth (sub-pulse duration): 0.001-5 ms inclusive, preferably 0.1-1 ms inclusive, and in some embodiments the sub-pulse duration may vary within a sub-pulse group, e.g., a first sub-pulse may have a longer duration than a second sub-pulse in the sub-pulse group;
Interval between micropulses (sub-pulse interval): 0-10 ms inclusive, 0.01-5 ms inclusive, preferably 0.01-1 ms inclusive;
Average power depends on the anatomical location, and irrigation and aspiration rates, typically in a range of 2-120 W inclusive, preferably 5 to 15 W (inclusive) in the ureter, 2-40 W (inclusive) in the kidney, and 5-40 W and 5-60 W (inclusive) in bladder;
Pulse repetition interval: in a range of 1-1000 ms (inclusive);
Pulse repetition rate (within the sub-pulse group): in a range of 0.5-500 Hz (inclusive);
Peak power (of sub-pulse in the sub-pulse group): in a range of 250-20,000 W (inclusive), preferably in a range of 500-5000 W inclusive, and in some embodiments the peak power may vary within the sub-pulse group, e.g., a first sub-pulse may have a lower or higher peak power than a second sub-pulse in the sub-pulse group.

### Maximizing Efficiency of Stone Fragmentation Procedures (Fragmentation Operational Mode)

The primary goal of stone fragmentation procedures is to break the stone into small enough fragments to be extracted by a basket or other tool, e.g. aspiration (usually, for fragments that are ~3 mm or smaller in size), especially when a rigid instrument is used. Based on theoretical modeling, experimental work and clinical tests performed by Applicant, a new mechanism of stone fragmentation was proposed and proven and is disclosed herein. To this end, in accordance with at least one embodiment an optimal scenario of the treatment is: (1) first create a temperature gradient in the stone to generate mechanical stress in the stone structure by slow release of the residual laser energy transferred to heating energy and, (2) then apply a strong thermo-mechanical impact through a shorter, high-energy pulse on the stressed stone structure to produce cracks (typically, along the one-dimensional line defects (dislocations), two-dimensional surface defects (grain, microcrystal cluster or crystalline domains, boundaries and free surfaces), and three-dimensional volume defects (pores), or other surfaces separating regions of the stone with different structure, eventually leading to stone breakage. In certain embodiments this can be achieved with a laser pulse consisting of two parts: a first (initial) group of sub-pulses for generating mechanical stress (in a target calculi), and a second (subsequent) group of sub-pulse for generating a thermo-mechanical impact, i.e., cracking of the mechanically stressed stone (the target calculi). As used herein, the term “mechanical stress” refers to structural stresses within the calculi that result from pressure variations or thermal gradients within the calculi, and such stresses can lead to fragmentation of the calculi itself. As used herein, the term “thermo-mechanical impact” refers to calculi fragmentation caused by thermal strain energy and stress, which is developing much faster during interaction with the first group of sub-pulses (as described in further detail below).

In accordance with one embodiment, the technique is to initially pump sufficient laser energy into the stone without causing substantial ablation, and then follow with a strong pulse to deliver the aforementioned thermo-mechanical impact. It should be noted that the retropulsion effect is of lesser significance for the fragmentation mode than for the dusting mode, since typically only large enough stones (~8 mm or larger) are targeted for fragmentation. In accordance with the fragmentation modes described herein, according to at least one embodiment a size of a fragment of the calculi after being exposed to the pulsed laser energy is at least 1 mm in a maximum cross-sectional dimension (which in the case of a circular fragment, would be a diameter), and in some embodiments a size of the fragment of the calculi after being exposed to the pulsed laser energy is in a range of 1-5 mm in a maximum cross-sectional dimension, and in preferred embodiment is in a range of 1-3 mm in a maximum cross-sectional dimension.

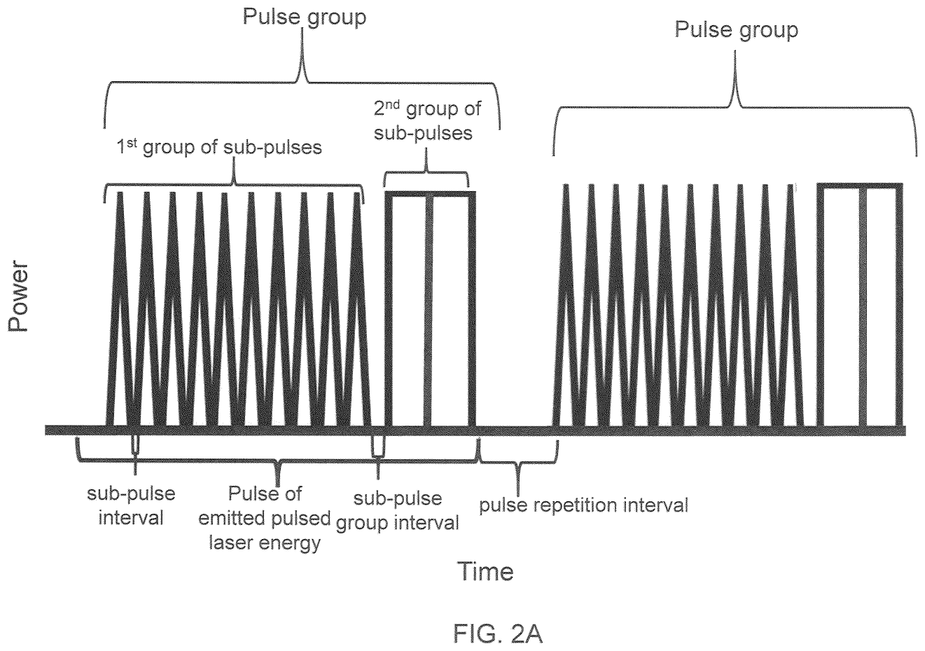

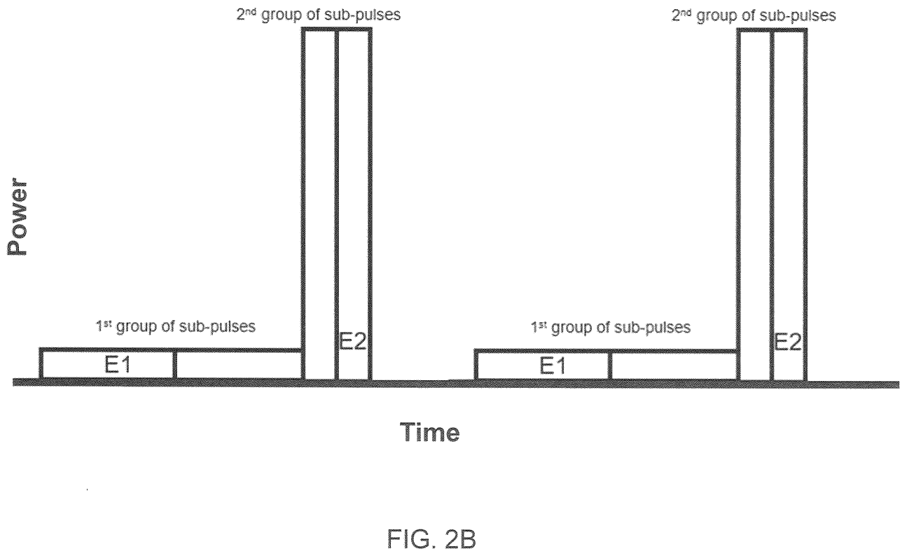

In one embodiment, the desired effect can be achieved through applying a first group (1 to 100) of sub-pulses where each sub-pulse of the first group of sub-pulses has a relatively small energy, and the sub-pulse interval (interval between sub-pulses) is short enough to prevent water (fluid) penetration on the treatment area of calculi during the sub-pulse interval that functions to cool the stone structure. An energetic pulse is then applied (which for the sake of simplicity may be referred to herein as a second group of sub-pulses) at a higher peak power. Therefore, in some embodiments, the emitted pulsed laser energy comprises two groups of sub-pulses separated in time by a sub-pulse group interval. In certain embodiments, the first group of sub-pulses has a sub-pulse group interval of a duration to prevent fluid penetration of an area of the target calculi that has been exposed to the pulsed laser energy. In some embodiments, each group of sub-pulses includes at least two sub-pulses separated in time by a sub-pulse interval that is in a range of 0-1 ms (inclusive). A sub-pulse interval with a value of 0 indicates that the two sub-pulses are joined (e.g., see first group of sub-pulses in FIG. 2B) and can be considered in some instances as one sub-pulse. The second group of sub-pulses may also have a sub-pulse interval that is zero, indicating the sub-pulses are joined (e.g., see second group of sub-pulses in FIG. 2B). In accordance with some embodiments, the first and second groups of sub-pulses can be grouped together into a pulse group, which can be emitted sequentially, and the pulse groups are separated by a pulse repetition interval, as shown in FIG. 2A.

One non-limiting example of a fragmentation mode for treating calculi is shown in FIG. 2A. During the first group of sub-pulses phase of stone treatment with this treatment, residual heating energy from every sub-pulse is propagated inside the stone structure to create non-uniform temperature distribution and mechanical stress around the treatment area where the laser pulse propagates and is absorbed by the stone. In accordance with at least one embodiment, the first group of sub-pulses has sufficient total energy (i.e., combined energy of all the sub-pulses included in the first group of sub-pulses), total pulse length (i.e., combined pulse length (duration) of all the sub-pulses in the first group of sub-pulses), and an average power (i.e., average power of the sub-pulses included in the first group of sub-pulses) to generate mechanical stress in a target calculi. If the energy of each individual sub-pulse is below the threshold of ablation, only the phenomenon of mechanical stress occurs. If the energy of each individual sub-pulse is above the threshold of ablation, then dusting occurs in addition to stone ablation. In accordance with at least one embodiment, the second group of sub-pulses has sufficient total energy (i.e., combined energy of all the sub-pulses included in the second group of sub-pulses), total pulse length (i.e., the combined pulse length (duration) of all the sub-pulses in the second group of sub-pulses), and an average power (i.e., average power of the sub-pulses included in the second group of sub-pulses) to generate a thermo-mechanical impact in the target calculi. This latter effect is explained in more detail below. The combined effect allows for the ability to fracture target calculi into fragments that are larger than 1 mm.

In accordance with an alternative embodiment, the group of sub-pulses can be replaced by a single pulse of sufficiently low peak power and long duration with power below or insignificantly (<1.5 times) above the threshold of ablation. In some embodiments, the target calculi has an ablation threshold and the energy of the first group of sub-pulses does not exceed the ablation threshold. In another embodiment, the energy of the first group of sub-pulses does not exceed the ablation threshold multiplied by a factor of 1.5.

One non-limiting example of another fragmentation mode of treating calculi is shown in FIG. 2B. Pre-heating via the first group of sub-pulses with a lower power results in an increase of the mechanical stress around the laser irradiated stone area results in an increase in the stone or mineral or organic component absorption coefficient of the stone matrix due to heating above 100-250° C. The second group of sub-pulses with the higher power will be more effectively absorbed by the stone material and produce more efficient thermal mechanical damage and stress due to better absorption when compared to the first group of sub-pulses. The higher peak power of the second group of sub-pulses in combination with the initial mechanical stress in the stone around the laser irradiated area results in the increased probability that the stone will crack into large parts.

In accordance with both approaches described above, in certain embodiments the described two groups of sub-pulses shape structure may be configured with high energy (up to 70 J) and a low rep rate (0.5-5 Hz). Applicant found that such higher energy pulses were also less damaging when accidentally hitting soft tissue in comparison with “regular” lower energy pulses with higher repetition rates and the same average power as the disclosed two groups of sub-pulses. This makes the disclosed stone treatment safer in comparison with conventional laser operating parameters used in stone fragmentation.

In accordance with at least one embodiment, optimal laser parameters for a fragmentation operational mode using pulse shape modulation as described above are as follows:
Total pulse energy including first and second groups of sub-pulses is 2-70 J inclusive, preferably 2-4 J inclusive for ureter stone and 7-60 J inclusive for kidney and bladder stone
Energy of first group of sub-pulses is 1-65 J inclusive, in some embodiments is 6.5-65 J inclusive, in some embodiments is 1-3 J inclusive for ureter stone and 7-60 J inclusive for kidney and bladder stone
Energy of second group of sub-pulses is 1-10 J inclusive, in some embodiments is 1-6.5 J inclusive, in some embodiments is 1-3 J inclusive for ureter stone and 3-10 J inclusive for kidney and bladder stone
Average power for first group of sub-pulses is 50-200 W inclusive
Peak power of second group of sub-pulses of pulse is 500-20,000 W inclusive, preferably 500-1500 W inclusive
Repetition rate of the two groups of sub-pulses is 0.5-10 Hz inclusive, preferably 0.5-5 Hz inclusive for ureter stone and 0.5-2 Hz for kidney or bladder stone

### Oscillations

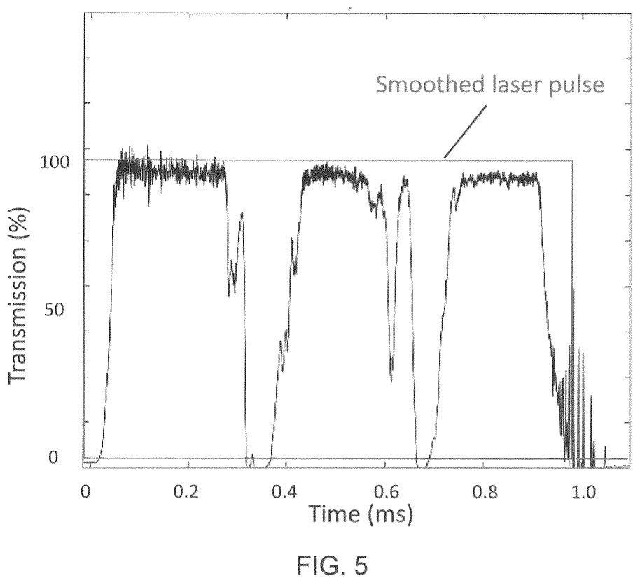

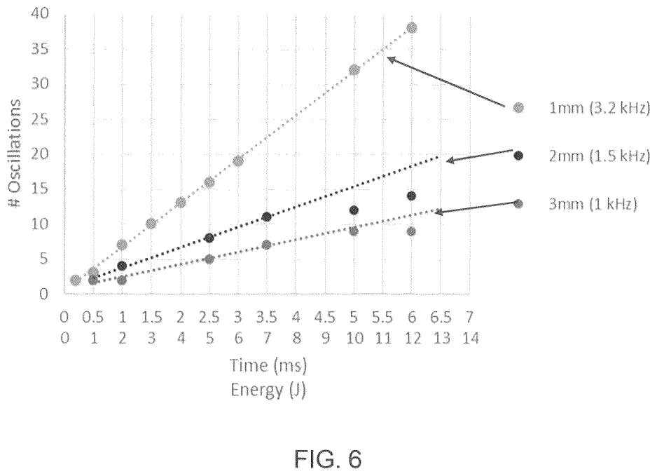

It is conventionally known that during a laser pulse in water, vaporization of water occurs leading to formation of a vapor channel between the fiber distal end and the stone surface (Moses channel). Experiments conducted by Applicant have revealed that the channel first expands in size and creates a vapor channel from the distal end of the fiber to the stone surface and then collapses. After collapse, another channel is generated during the laser pulse and this process repeats throughout the duration of the laser pulse. This phenomena as recognized by Applicant is referred to as oscillation of the vaporization (Moses) channel. This channel oscillation occurs because the laser energy input to create a channel stops when the channel is formed and this energy is stored in the surrounding water as increased potential energy. This potential energy relaxes to collapse the channel. Coupling of laser energy into the collapsing water begins the vaporization process again leading to the formation of the next channel. This process repeats over the duration of the laser pulse. The experiments conducted by the Applicant have shown that during the laser pulse, oscillation of the vapor (Moses) channel causes the transmission of laser power to the stone to also oscillate during the laser pulse. FIG. 5 shows the transmission of laser power (black curve) through the oscillating vapor channel when the fiber-stone distance is 0.8 mm and the laser power (labeled in figure) is 500 W for a 1 ms pulse duration (TFL laser used, peak power of 500 W and 1 ms pulse duration). The number of transmission oscillations increases linearly with pulse duration at a rate that depends on fiber-stone distances, as shown in FIG. 6.

### Reduction of Retropulsion (Operational Mode)

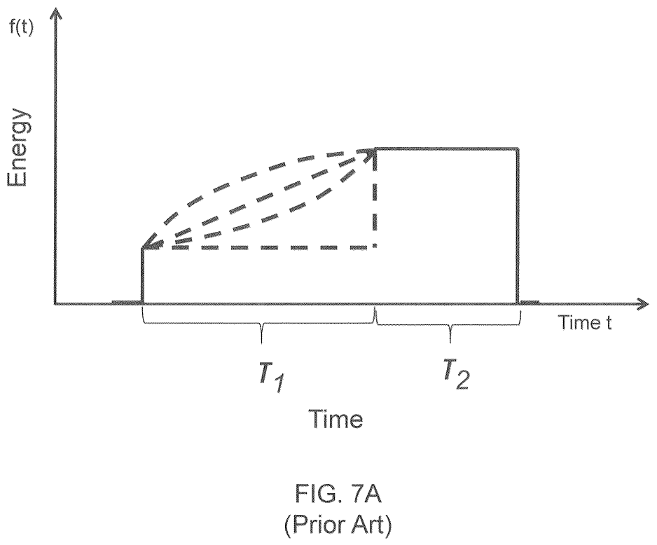

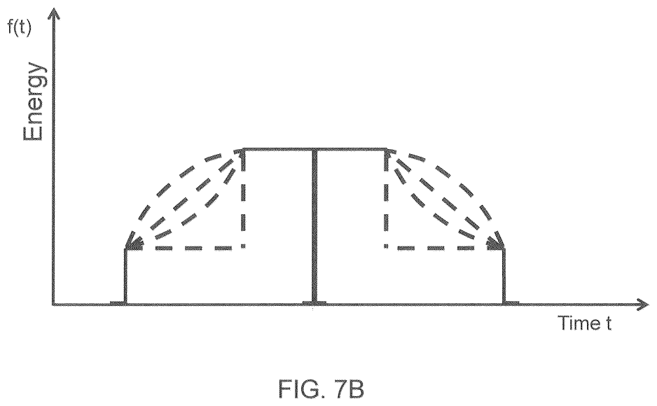

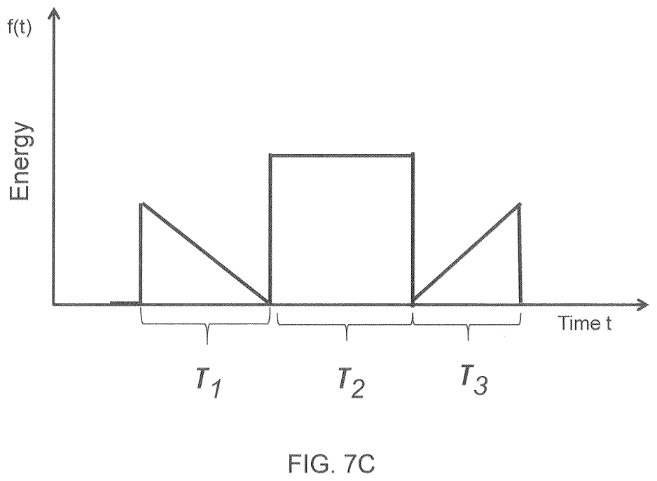

The flow of water during the channel formation and expansion, the vapor pressure within the channel, and the flow of water during and after channel collapse are all forces that act upon the stone at different times. The net result is stone oscillation movements that occur synchronized with channel oscillations. Applicant refers to this as a “trapping” effect. Experiments performed by Applicant have demonstrated that the amplitude of stone oscillations during the trapping effect are much less than 1 mm for stone sizes around 5 mm. Smaller/larger amplitudes of oscillations are expected for larger/smaller stones, respectively. The non-oscillatory movement of the stone after the laser pulse depends on the character of and the timing of the final channel collapse. Final channel collapse produces flow of water that is the primary source of unwanted stone retropulsion. The direction of the water flow depends on the conditions of the final channel, namely, the channel shape and the velocity of and pressure difference across its boundaries. For example, a symmetrical channel collapse generates no net fluid momentum that can be transferred to the stone. Conversely, an appropriately formed channel can collapse to provide negative/positive stone movement toward/away from the fiber, respectively. The state of the final channel can be controlled by the laser power and pulse profile. The use of a combination of two sub-pulses to reduce retropulsion is known in the art, an example of which is shown in FIG. 7A, which is from PCT Application No. PCT/US 2019/042491 and published as WO 2020/033121 and is owned by Applicant and incorporated herein by reference in its entirety. The first sub-pulse or portion is used for initiating channel formation between the fiber and the stone, ablation of the stone, and preheating of the stone around ablation crater, depending on distance of fiber to stone. Recent experiments by Applicant have demonstrated that the first sub-pulse can further reduce retropulsion if its duration is decreased to a fraction of a single channel period. The retropulsion effect of such pulsing will be decreased because a more symmetrical channel and channel collapse occurs and there is less momentum transfer to the fluid. In FIG. 7A, T₁ is the duration of the first sub-pulse or portion and T₂ is the duration of the second sub-pulse or portion. In addition, the energy or power profile of the first sub-pulse or portion can be constant on a level Pₘᵢₙ or take on the form of a function, such as linear, exponential, or polynomic functions that increase from Pₘᵢₙ to Pₘₐₓ, where Pₘₐₓ is the peak power of the second sub-pulse or portion. Recent experiments by Applicant have demonstrated that further reduction in retropulsion occurs when the power of the first sub-pulse is low and its duration is synchronized with the channel or bubble frequency.

Recent studies performed by Applicant have also demonstrated the importance not only of the front edge of the pulse, but also of a trailing edge. In particular, the rate of collapse of the final Moses channel can be decreased with a lower power setting that delays final collapse and lessens the collapsing water flow velocity. This is achieved through introduction of an additional post-sub-pulse, at reduced power levels, thus creating a “triple” pulse, i.e., three sub-pulses. In some embodiments, a controller is configured to adjust the laser power profile such that each emitted pulse energy comprises three sub-pulses. One non-limiting example of such a “triple” pulse is shown in FIG. 7B. As indicated in FIG. 7B, the first sub-pulse (energy) may increase in accordance with any of a linear, polynomial, and exponential function, and the third sub-pulse may decrease in accordance with any of a linear, polynomial, and exponential function. In addition, the third sub-pulse (energy, power) can be constant on a level Pₘᵢₙ. Furthermore, some embodiments of (the energy, power) of the “triple” pulse may involve use of inversely sloped sub-pulses, one non-limiting example of which is shown in FIG. 7C. For example, in some embodiments respective slopes of the energy of the first and third sub-pulses are inverses of one another. In yet other embodiments, the post-sub-pulse format may comprise a sequence of sub-pulses that are chirped (with reducing pulse duration and pulse power) to match the decreasing Moses' channel diameters and their duration (proportional to the channel diameters under given ambient pressure).

In accordance with at least one embodiment, in order to reduce retropulsion and yet ensure efficacy of the pulse for the intended purpose (dusting, fragmentation, etc.), the following laser parameters are optimal for the pre-(1^{st}) and post-(3^{rd}) sub-pulses:
peak power in a range of 50-200 W inclusive, preferably in a range of 100-130 W inclusive
pulse energies in a range of 0.005-1 J inclusive, preferably in a range of 0.01-0.3 J inclusive
1^{st} sub-pulse durations are in a range of 100 microseconds-1 ms (inclusive)
   In accordance with some embodiments, the following laser parameters may be used in the reduced retropulsion operational mode:
second sub-pulse has a higher energy than the first and third sub-pulses
second sub-pulse energy in a range of 0.01-10 J inclusive, in a range of 0.1-3 J inclusive
second sub-pulse peak power in a range of 400-4000 W inclusive, preferably 400-1500 W inclusive
sub-pulse interval (time duration in between sub-pulses) is in a range of 0-0.5 ms inclusive
sub-pulse interval is in a range of 0-300 μs
sub-pulse duration in a range of 0.1-1 ms

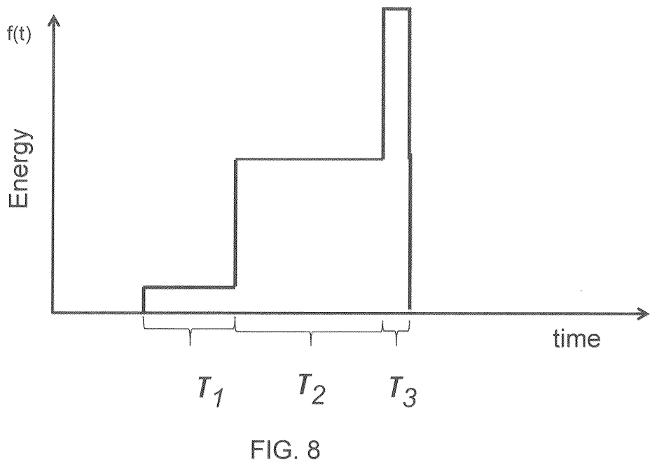

An additional embodiment encompasses a concept where the laser power is increased at the end of the pulse as shown in FIG. 8, which results in the restoration of the channel occurring sooner. Therefore, more energy will be transferred to the stone for ablation per pulse.

For stones that have moved away from the fiber (at distances>2 mm) the pulse profile shown in FIG. 8 may be applied to draw the stone back toward the fiber (negative retropulsion). Applicant refers to this process as stone “retraction.” This occurs because the final Moses channel collapses in a way that causes water to flow toward the fiber.

In accordance with these embodiments, the following laser parameters may be used in the enhanced ablation efficiency/retraction operational modes:
first sub-pulse has a lower energy than the subsequent sub-pulses
second sub-pulse energy at intermediate power level for channel vaporization in a range of 0.01-10 J inclusive, in a range of 0.1-3 J inclusive
third sub-pulse is at highest power level to generate higher pressure at distal end of the channel (near stone) for asymmetrical collapse greater at distal end than at proximal end (collapse toward fiber).

Stones smaller in size than 5 mm can be trapped and oscillated during the laser pulse. The amplitude of stone oscillations can be in the range of 0.1-3 mm, and the frequency of Moses channel oscillation is in the range of 0.5-10 kHz, depending on the average fiber-stone distance. The above embodiments are methods to reduce or maintain the stone's average distance to the fiber with a corresponding increase in ablation efficiency. Additional methods to both increase ablation efficiency and reduce retropulsion allow for laser pulse power modulation synchronized with Moses channel oscillations (FIG. 8; as well as to decrease amplitude of stone oscillation to the range below 1 mm and preferable below 0.5 mm. This will increase ablation efficiency.

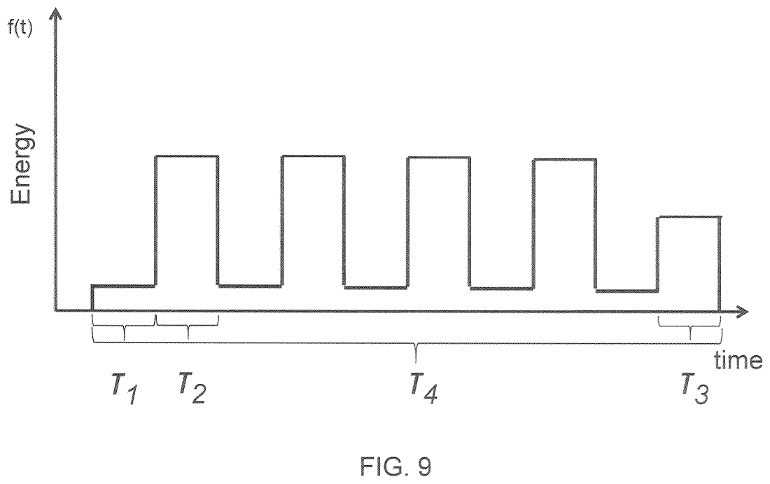

Applicant has observed that there are three phases during the laser pulse that are important: Phase 1, of duration T₁, is the optimum power and duration for onset of channel formation affecting shape and dynamics of the channel near the fiber. Phase 2, of duration T₂, is the optimum power and duration for stone ablation (through the fully developed channel). Phase 3, of duration T₃, is the optimum power and pulse duration for the final channel collapse at the end of the laser pulse. Therefore, power during the laser pulse may be modulated and synchronized to channel oscillations to increase ablation efficiency as well as to reduce retropulsion simultaneously. A non-limiting example of such a pulse configuration is shown in FIG. 9. The period of time T₁ is set at a power to affect the state of the Moses channel at the fiber end, T₂ is set at high power for best stone ablation rate and T₃ is set at a power level to obtain symmetric channel conditions from the stone end to the fiber end for reduction of retropulsion.

In accordance with these embodiments, the following laser parameters may be used in this operational mode:
Duration T₁ in the range 0.050-2 ms with power in a range of 50-250 W inclusive, preferably in a range of 100-130 W inclusive
Duration T₂ in the range 0.050-2 ms with power in a range of 50-4000 W inclusive
Duration T₃ in the range 0.050-2 ms with power in a range of 50-1000 W inclusive, pulse energies in a range of 0.005-1 J inclusive
Duration T₄ in the range 1-6 ms with power in a range of 50-4000 W inclusive

### Laser System

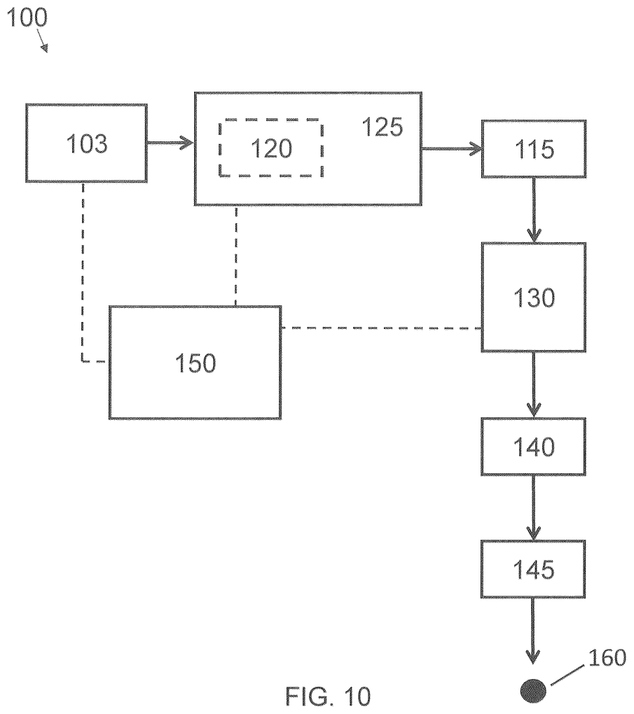

FIG. 10 is a block diagram that shows one non-limiting example of a laser system configured to generate the laser pulse operational modes described above. It is to be appreciated that other configuration may be used to implement the aforementioned pulse modes.

Laser system 100 comprises a powers supply 103, a laser driver 125 that may include an optional energy storage device 120, a pump 115, a laser module 130, and a controller 150, also referred to herein as a control module. Laser energy from the laser module 130 is directed to the target calculi 160. Laser system 100 may also include a beam delivery system or module 145 and an optical coupler 140.

The pump 115 is configured with one or more diode lasers that energy laser 130. The power supply 103 supplies power to the system and optional energy storage device 125 (e.g., electrical capacitor and/or inductor) can be configured to store a sufficient amount of energy necessary to form a laser pulse. The laser driver 125 of the pump 115 forms an electrical pulse of specified characteristics in response to a control signal from control module 150. The electrical pulses are received by one or more diodes of pump 115 which form an optical pulse necessary to pump the laser medium in laser module 130. The output of the laser module 130 is coupled to a beam delivery system 145, and in some instances this coupling occurs via optical coupler 140.

One or more components of the laser system 100 are controlled by controller 150, which is programmed with control signals that are used to control the laser driver 125, power supply 103, and/or laser module 130. For instance, the control signal from controller 150 can be used to directly modulate the pump current of driver 125 used to pump diodes 115, as will be appreciated by those skilled in the art, to output the desired pulse energy, power, and temporal structure. In some embodiments, a single charge-discharge cycle of the energy storage device 120 can be used to modulate the diode current.

The aspects disclosed herein in accordance with the present invention, are not limited in their application to the details of construction and the arrangement of components set forth in the following description or illustrated in the accompanying drawings. These aspects are capable of assuming other embodiments and of being practiced or of being carried out in various ways. Examples of specific implementations are provided herein for illustrative purposes only and are not intended to be limiting. In particular, acts, components, elements, and features discussed in connection with any one or more embodiments are not intended to be excluded from a similar role in any other embodiments.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. In addition, in the event of inconsistent usages of terms between this document and documents incorporated herein by reference, the term usage in the incorporated reference is supplementary to that of this document; for irreconcilable inconsistencies, the term usage in this document controls. Moreover, titles or subtitles may be used in the specification for the convenience of a reader, which shall have no influence on the scope of the present invention.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description and drawings are by way of example only.

1. A laser system for treating calculi, comprising:
a laser configured to emit pulsed laser energy having a wavelength a range of 1.85 to 2.2 microns (μm) inclusive; and
a controller configured to control the laser such that the pulsed laser energy is emitted as a sequence of sub-pulse groups separated in time by a pulse repetition interval, and each sub-pulse group includes at least two sub-pulses separated in time by a sub-pulse interval that is in a range of 0-10 milliseconds (ms), wherein
each sub-pulse of the sub-pulse group has a pulse duration in a range of 0.001-5 ms inclusive,
each sub-pulse of the sub-pulse group has an energy in a range of 0.001-1 Joules (J) inclusive, and
the pulse repetition interval is in a range of 1-1000 ms inclusive.

2. The laser system of claim 1, wherein the sub-pulse group includes 2-1000 sub-pulses.

3. The laser system of claim 2, wherein the sub-pulse group includes 2-100 sub-pulses.

4. The laser system of claim 3, wherein the sub-pulse group includes 2-10 sub-pulses.

5. The laser system of claim 1, wherein a total energy of the sub-pulse group is in a range of 0.2-5 J inclusive.

6. The laser system of claim 6, wherein the total energy of the sub-pulse group is in a range of 0.5-2 J inclusive.

7. The laser system of claim 1, wherein the energy of each sub-pulse is in a range of 0.01-1 J inclusive.

8. The laser system of claim 7, wherein the energy of e pulse is in a range of 0.2-1 J inclusive.

9. The laser system of claim 1, wherein the sub-pulse interval is in a range of 0.01-5 ms inclusive.

10. The laser system of claim 9, wherein the sub-pulse interval is in a range of 0.01-1 ms inclusive.

11. The laser system of claim 1, wherein a duration of each sub-pulse is in a range of 0.1-1 ms inclusive.

12. The laser system of claim 1, wherein the pulsed laser power in a range of 2-120 Watts (W) inclusive,

13. The laser system of claim 12, wherein the average power is in a range of 2-40 W inclusive.

14. The laser system of claim 13, wherein the average power is in a range of 5-40 W inclusive.

15. The laser system of claim 1, wherein the pulse repetition rate within the sub-pulse group is in a range of 0.5-500 Hz inclusive.

16. The laser system of claim 1, wherein a peak power of a sub-pulse of the sub-pulse group is in a range of 250-20000 W inclusive.

17. The laser system of claim 16, wherein the peak power of the sub-pulse of the sub-pulse group is in a range of 0-5000 W inclusive.

18. The last system of claim 1, wherein at least one sub-pulse of the sub-pulse group has a pulse shape such that a power of the at least one sub-pulse monotonically increases from a beginning of the sub-pulse to an end of the sub-pulse.

19. The laser system of claim 1, wherein the laser is a thulium fiber laser, a thulium solid state laser, or a holmium solid state laser.

20-71. (canceled)
